(19) United States
(12) Patent Application Publication
(10) Pub. No.: US 2023/0012841 A1
(43) Pub. Date: Jan. 19, 2023

(54) EXCRETA DETERMINATION METHOD, EXCRETA DETERMINATION DEVICE, AND NON-TRANSITORY COMPUTER READABLE RECORDING MEDIUM STORING EXCRETA DETERMINATION PROGRAM

(71) Applicant: PANASONIC HOLDINGS CORPORATION, Osaka (JP)

(72) Inventors: Yuka YAMADA, Nara (JP); Hirofumi KANAI, Osaka (JP); Toshihide MORI, Osaka (JP); Takahiro KAMAI, Kyoto (JP)

(73) Assignee: PANASONIC HOLDINGS CORPORATION, Osaka (JP)

(21) Appl. No.: 17/948,695

(22) Filed: Sep. 20, 2022

Related U.S. Application Data
(63) Continuation of application No. PCT/JP2020/048946, filed on Dec. 25, 2020.

(30) Foreign Application Priority Data
Nov. 12, 2020 (JP) 2020-188528

(51) Int. Cl. G01N 33/497; E03D 9/00

(52) U.S. Cl. CPC G01N 33/497; E03D 9/00; G01N 2033/4975

(57) ABSTRACT

An excreta determination device includes a data acquisition unit that acquires first time series data indicating hydrogen concentration in a space inside a toilet bowl measured by an internal sensor arranged inside the toilet bowl, an excreta determination unit that determines whether or not one who excreted has farted based on the first time series data of the hydrogen concentration, and a determination result output unit that outputs a determination result, and the excreta determination unit determines that the one who excreted has farted when the value of the hydrogen concentration in the first time series data exceeds a threshold value.

### TECHNICAL FIELD

The present disclosure relates to a technique for determining excreta.

### BACKGROUND ART

The presence or absence of excreta such as feces, urine, and fart, the type of excreta, the number of times of excreting, and the time of excreting are important information for managing the health of the care receiver. The caregiver records information on excreta of the care receiver, but the recording of the information is a burden on the caregiver and the care receiver. In addition, when recording information regarding excreta by a report from a care receiver, it is difficult to obtain accurate information regarding excreta from a care receiver with dementia.

Therefore, conventionally, an excretion management system that objectively manages excretion has been desired. For example, the excretion management system of Patent Literature 1 includes a temperature measurement part for measuring the space distribution of the temperature in a bowl part of a toilet bowl in noncontact and a control part that determines the presence or absence of excretion in the bowl part based on the temperature data. In addition, the conventional excretion management system further includes an odor measurement part for measuring an odor in the bowl part. Based on the odor data measured by an odor measurement part and the temperature data, the control part discriminates at least one of feces, urine, and fart regarding excreta excreted in the bowl part.

However, in the above-described conventional technique, it is difficult to accurately determine whether or not one who excreted has farted, and further improvement has been required.

### CITATION LIST

### Patent Literature

Patent Literature 1: Japanese Patent Application Laid-Open No. 2015-178764

### SUMMARY OF INVENTION

The present disclosure has been made to solve the above problem, and an object of the present disclosure is to provide a technique capable of accurately determining whether or not one who excreted has farted.

In an excreta determination method according to an aspect of the present disclosure, a computer acquires first time series data indicating hydrogen concentration in a space inside a toilet bowl measured by an internal sensor arranged inside the toilet bowl, determines whether or not one who excreted has farted based on the first time series data of the hydrogen concentration, and outputs a determination result.

According to the present disclosure, it is possible to accurately determine whether or not one who excreted has farted.

### BRIEF DESCRIPTION OF DRAWINGS

FIG. 1 is a view illustrating a configuration of an excretion management system in a first embodiment of the present disclosure.

FIG. 2 is a view for describing arrangement positions of an internal sensor and an excreta determination device in the first embodiment of the present disclosure.

FIG. 3 is a flowchart for describing excreta determination processing in the excreta determination device according to the first embodiment of the present disclosure.

FIG. 4 is a view illustrating an example of first time series data of hydrogen concentration in a case where urination and defecation are present and fart is not present in the present first embodiment.

FIG. 5 is a view illustrating an example of first time series data of hydrogen concentration in a case where urination and fart are present and defecation is not present in the present first embodiment.

FIG. 6 is a view illustrating a configuration of an excretion management system in a second embodiment of the present disclosure.

FIG. 7 is a view for describing arrangement positions of an internal sensor, an external sensor, and an excreta determination device in the second embodiment of the present disclosure.

FIG. 8 is a flowchart for describing excreta determination processing in the excreta determination device according to the second embodiment of the present disclosure.

FIG. 9 is a view illustrating an example of first time series data and second time series data of hydrogen concentration in a case where urination and fart are present and defecation is not present in the present second embodiment.

FIG. 10 is a view illustrating an example of first time series data and second time series data of hydrogen concentration in a case where urination and defecation are present and fart is not present in the present second embodiment.

FIG. 11 is a view illustrating a configuration of an excretion management system in a third embodiment of the present disclosure.

FIG. 12 is a flowchart for describing excreta determination processing in the excreta determination device according to the third embodiment of the present disclosure.

FIG. 13 is a view illustrating an example of first time series data of hydrogen concentration in a case where farting is present and defecation is not present in the present third embodiment.

FIG. 14 is a view illustrating an example of first time series data of hydrogen concentration in a case where defecation is present and farting is not present in the present third embodiment.

FIG. 15 is a view illustrating a configuration of an excretion management system in a fourth embodiment of the present disclosure.

FIG. 16 is a first flowchart for describing excreta determination processing in the excreta determination device according to the fourth embodiment of the present disclosure.

FIG. 17 is a second flowchart for describing excreta determination processing in the excreta determination device according to the fourth embodiment of the present disclosure.

FIG. 18 is a view illustrating an example of first time series data of hydrogen concentration in a case where farting is present and defecation is not present in the present fourth embodiment.

### DESCRIPTION OF EMBODIMENTS

(Knowledge Underlying Present Disclosure)

In the above-described conventional excretion management system, the control part compares the temperature data T_{i,ch} at each time with the temperature threshold value Tₙ for the temperature time series data, determines that excretion is not performed when T_{i,ch}≤Tₙ, and determines that excretion is performed when T_{i,ch}>Tₙ.

The odor measurement part includes a hydrogen sulfide odor sensor having high sensitivity to a hydrogen sulfide odor and an ammonia odor sensor having high sensitivity to an ammonia odor. The control part compares the odor data O₁i at each time in the odor time series data of the hydrogen sulfide odor sensor with the first odor threshold value Oₙ₁, and compares the odor data O 2ᵢ at each time in the odor time series data of the ammonia odor sensor with the second odor threshold value Oₙ₂. When the temperature data T_{i,ch} at each time of the temperature time series data is T_{i,ch}>Tₙ, that is, when excretion is performed, the control part determines that the excretion is feces if O₁ᵢ>Oₙ₁ or O₂ᵢ>Oₙ₂, and determines that the excretion is urine if O₁ᵢ≤Oₙ₁ and O₂ᵢ≤Oₙ₂. In addition, when the temperature data T_{i,ch} at each time of the temperature time series data is T_{i,ch}≤Tₙ, that is, when excretion is not performed, the control part determines that fart has been performed if O₁ᵢ>Oₙ₁ or O₂ᵢ>Oₙ₂.

As described above, based on temperature data, odor data of the hydrogen sulfide odor sensor, and odor data of the ammonia odor sensor, the conventional technique discriminates as to which of feces, urine, or fart the excreta is.

However, the concentration of the hydrogen sulfide component contained in the gas excreted from the anus of one who excreted changes depending on the physical condition such as constipation of the one who excreted. In addition, the concentration of the hydrogen sulfide component contained in the excreted gas greatly changes depending on the food eaten by the one who excreted, and also greatly changes depending on the medicine taken.

Therefore, it is difficult to accurately determine whether or not the one who excreted has farted using the concentration of the hydrogen sulfide component contained in the excreta.

In order to solve the above problems, in an excreta determination method according to an aspect of the present disclosure, a computer acquires first time series data indicating hydrogen concentration in a space inside a toilet bowl measured by an internal sensor arranged inside the toilet bowl, determines whether or not the one who excreted has farted based on the first time series data of the hydrogen concentration, and outputs a determination result.

The hydrogen concentration contained in the gas excreted from the anus of the human body is hardly affected by the food eaten by the human body and the medicine taken by the human body. Therefore, it is possible to accurately determine whether or not the one who excreted has farted by determining whether or not the one who excreted has farted based on the first time series data indicating the hydrogen concentration in the space inside the toilet bowl.

Furthermore, in the above excreta determination method, in the determination, when the value of the hydrogen concentration in the first time series data exceeds a threshold value, it may be determined that the one who excreted has farted.

When the one who excreted farts into the toilet bowl, the hydrogen concentration in the space inside the toilet bowl increases. Therefore, by determining whether or not the value of the hydrogen concentration in the first time series data has exceeded the threshold value, it is possible to easily determine whether or not the one who excreted has farted.

Furthermore, in the excreta determination method, in the determination, it may be determined that the one who excreted has farted when the value of the hydrogen concentration in the first time series data exceeds a threshold value, a rising slope of the first time series data until reaching a peak is larger than a threshold value, and a falling slope of the first time series data after reaching the peak is smaller than a threshold value.

The gas excreted into the toilet bowl does not stay in the toilet bowl but is diffused to the outside of the toilet bowl. Therefore, when the one who excreted farts into the toilet bowl, the first time series data of the hydrogen concentration in the space inside the toilet bowl rises sharply, reaches a peak, and then falls sharply. Therefore, it can be determined that the one who excreted has farted when the value of the hydrogen concentration in the first time series data exceeds a threshold value, a rising slope of the first time series data until reaching a peak is larger than a threshold value, and a falling slope of the first time series data after reaching the peak is smaller than a threshold value.

Furthermore, in the above excreta determination method, second time series data indicating hydrogen concentration in a space outside the toilet bowl measured by an external sensor arranged outside the toilet bowl may be further acquired, and in the determination, it may be determined whether or not the one who excreted has farted based on the first time series data and the second time series data.

The gas excreted into the toilet bowl does not stay in the toilet bowl but is diffused to the outside of the toilet bowl. Therefore, by measuring the hydrogen concentration not only in the space inside the toilet bowl but also in the space outside the toilet bowl, it is possible to more accurately determine whether or not the one who excreted has farted.

Furthermore, in the excreta determination method, in the determination, it may be determined that the one who excreted has farted when the value of the hydrogen concentration in the first time series data exceeds a threshold value and the value of the hydrogen concentration in the second time series data exceeds a threshold value.

When the one who excreted farts into the toilet bowl, the hydrogen concentration in the space inside the toilet bowl increases. In addition, since the gas excreted into the toilet bowl is diffused to the outside of the toilet bowl, the hydrogen concentration in the space outside the toilet bowl also increases. Therefore, it is possible to more accurately determine whether or not the one who excreted has farted by determining whether or not the value of the hydrogen concentration in the first time series data has exceeded the threshold value and determining whether or not the value of the hydrogen concentration in the second time series data has exceeded the threshold value.

Furthermore, in the above excreta determination method, first time at which the first time series data reaches a peak may be further acquired, second time at which the first time series data converges after the first time series data reached the peak may be further acquired, and in the determination, it may be determined which of feces or fart the one who excreted has excreted based on the first time and the second time.

The gas excreted into the toilet bowl is diffused to the outside of the toilet bowl. Therefore, when the one who excreted farts into the toilet bowl, the first time series data of the hydrogen concentration in the space inside the toilet bowl falls sharply after reaching the peak. On the other hand, feces excreted into the toilet bowl remains in the toilet bowl. Therefore, when the one who excreted defecates into the toilet bowl, the first time series data of the hydrogen concentration in the space inside the toilet bowl gradually decreases after reaching the peak. Therefore, the elapsed time from the first time at which the first time series data reaches the peak to the second time at which the first time series data converges is different between fart and defecation. Therefore, based on the elapsed time from when the value of the hydrogen concentration reaches the peak to when it converges, it is possible to determine which of feces and fart the one who excreted has excreted.

Furthermore, in the excreta determination method, in the determination, it may be determined that the one who excreted has farted when a difference between the second time and the first time is equal to or less than a predetermined time, and it may be determined that the one who excreted has defecated when the difference is longer than the predetermined time.

The elapsed time from when the value of the hydrogen concentration in the space inside the toilet bowl reaches the peak to when it converges becomes longer when the one who excreted defecates into the toilet bowl than when the one who excreted farts into the toilet bowl. Therefore, by comparing the elapsed time from when the values of the hydrogen concentration reaches the peak to when it converges with the predetermined time, it is possible to determine which one of feces and fart the one who excreted has excreted.

Furthermore, in the above excreta determination method, second time series data indicating hydrogen concentration in a space outside the toilet bowl measured by an external sensor arranged outside the toilet bowl may be further acquired, third time at which the second time series data reaches a peak may be further acquired, fourth time at which the second time series data converges after the second time series data reached the peak may be further acquired, and in the determination, it may be determined which of feces or fart the one who excreted has excreted based on the first time, the second time, the third time, and the fourth time.

The gas excreted into the toilet bowl is diffused to the outside of the toilet bowl. Therefore, when the one who excreted farts into the toilet bowl, the second time series data of the hydrogen concentration in the space outside the toilet bowl falls sharply after reaching the peak, similarly to the first time series data. On the other hand, when the one who excreted defecates into the toilet bowl, the second time series data of the hydrogen concentration in the space outside the toilet bowl gradually decreases after reaching the peak, similarly to the first time series data. Therefore, it is possible to more accurately determine which of feces or fart the one who excreted has excreted based on the elapsed time from the first time at which the first time series data reaches the peak to the second time at which the first time series data converges and the elapsed time from the third time at which the second time series data reaches the peak to the fourth time at which the second time series data converges.

Furthermore, in the above excreta determination method, in the determination, it may be determined that the one who excreted has farted when a first difference between the second time and the first time is equal to or less than a predetermined time and a second difference between the fourth time and the third time is equal to or less than a predetermined time, and it may be determined that the one who excreted has defecated when the first difference is longer than the predetermined time and the second difference is longer than the predetermined time.

The elapsed time from when the value of the hydrogen concentration in the space inside the toilet bowl reaches the peak to when it converges becomes longer when the one who excreted defecates into the toilet bowl than when the one who excreted farts into the toilet bowl. Similarly, the elapsed time from when the value of the hydrogen concentration in the space outside the toilet bowl reaches the peak to when it converges is longer when the one who excreted defecates into the toilet bowl than when the one who excreted farts into the toilet bowl. Therefore, it is possible to more accurately determine which of feces or fart the one who excreted has excreted by comparing the elapsed time from when the value of the hydrogen concentration in the space inside the toilet bowl reaches the peak to when it converges with the predetermined time, and comparing the elapsed time from when the value of the hydrogen concentration in the space outside the toilet bowl reaches the peak to when it converges with the predetermined time.

An excreta determination device according to another aspect of the present disclosure includes: an acquisition unit that acquires first time series data indicating hydrogen concentration in a space inside a toilet bowl measured by an internal sensor arranged inside the toilet bowl; a determination unit that determines whether or not one who excreted has farted based on the first time series data of the hydrogen concentration; and an output unit that outputs a determination result.

The hydrogen concentration contained in the gas excreted from the anus of the human body is hardly affected by the food eaten by the human body and the medicine taken by the human body. Therefore, it is possible to accurately determine whether or not the one who excreted has farted by determining whether or not the one who excreted has farted based on the first time series data indicating the hydrogen concentration in the space inside the toilet bowl.

A non-transitory computer readable recording medium storing an excreta determination program according to another aspect of the present disclosure causes a computer to function to acquire first time series data indicating hydrogen concentration in a space inside a toilet bowl measured by an internal sensor arranged inside the toilet bowl, determine whether or not one who excreted has farted based on the first time series data of the hydrogen concentration, and output a determination result.

The hydrogen concentration contained in the gas excreted from the anus of the human body is hardly affected by the food eaten by the human body and the medicine taken by the human body. Therefore, it is possible to accurately determine whether or not the one who excreted has farted by determining whether or not the one who excreted has farted based on the first time series data indicating the hydrogen concentration in the space inside the toilet bowl.

Embodiments of the present disclosure will be described below with reference to the accompanying drawings. The following embodiments are examples of embodiment of the present disclosure, and are not intended to limit the technical scope of the present disclosure.

### First Embodiment

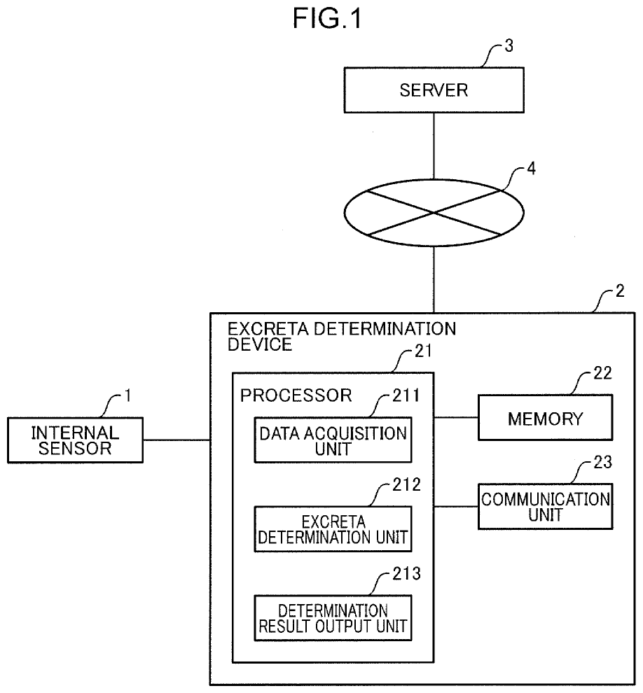

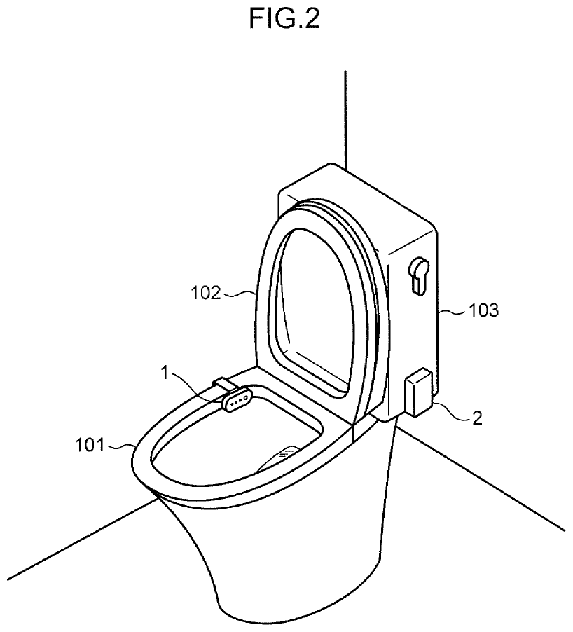

FIG. 1 is a view illustrating the configuration of an excretion management system in the first embodiment of the present disclosure. FIG. 2 is a view for describing the arrangement positions of an internal sensor 1 and an excreta determination device 2 in the first embodiment of the present disclosure.

The excretion management system illustrated in FIG. 1 includes an internal sensor 1, an excreta determination device 2, and a server 3.

The internal sensor 1 is arranged inside a toilet bowl 101 and has sensitivity to hydrogen. As illustrated in FIG. 2, the internal sensor 1 is hung on an edge of an opening formed in an upper part of the toilet bowl 101 that receives feces and urine. The internal sensor 1 measures hydrogen concentration in a space inside the toilet bowl 101. The internal sensor 1 is connected to the excreta determination device 2 communicably with each other in a wired or wireless manner. The internal sensor 1 transmits first time series data of the measured hydrogen concentration to the excreta determination device 2.

A bottom part of the toilet bowl 101 is provided with a drain channel not illustrated. Feces and urine excreted into the toilet bowl 101 are caused to flow through the drain channel. An upper part of the toilet bowl 101 is provided with a toilet seat 102 for the one who excreted to sit. The toilet seat 102 rotates up and down. The one who excreted sits down in a state where the toilet seat 102 is lowered onto the toilet bowl 101. A rear of the toilet bowl 101 is provided with a flush tank 103 that stores water for flushing feces and urine.

The internal sensor 1 may constantly transmit the first time series data of the measured hydrogen concentration to the excreta determination device 2. In addition, the internal sensor 1 may transmit, to the excreta determination device 2, the first time series data of the hydrogen concentration measured in a period from a time point at which the one who excreted sits on the toilet seat 102 to a time point at which the one who excreted leaves the toilet seat 102. For example, the toilet seat 102 may be provided with a pressure sensor, and whether or not the one who excreted sits on the toilet seat 102 may be determined based on output from the pressure sensor. Furthermore, determination as to whether the one who excreted sits on the toilet seat 102 may be made by using the fact that the inside of the toilet bowl 101 is darkened when the one who excreted sits on the toilet seat 102. That is, inside the toilet bowl 101 is provided with a light sensor, and when the light sensor detects that the toilet bowl is darkened, it may be determined that the one who excreted has sat on the toilet seat 102, and when the light sensor detects that the toilet bowl is brightened, it may be determined that the one who excreted left the toilet seat 102.

The excreta determination device 2 is arranged, for example, on a side surface of the flush tank 103. Note that the arrangement position of the excreta determination device 2 is not limited to the above, and may be any position as long as it is in the toilet. When the internal sensor 1 and the excreta determination device 2 are wirelessly connected to each other, the excreta determination device 2 needs not be arranged inside the toilet and only needs to be arranged at a place where wireless communication with the internal sensor 1 in the house is possible.

The excreta determination device 2 includes a processor 21, a memory 22, and a communication unit 23.

The memory 22 is a storage device capable of storing various types of information, such as a random access memory (RAM), a solid state drive (SSD), or a flash memory. The memory 22 stores the first time series data transmitted by the internal sensor 1.

The processor 21 is, for example, a central processing unit (CPU). The processor 21 implements a data acquisition unit 211, an excreta determination unit 212, and a determination result output unit 213.

The data acquisition unit 211 acquires the first time series data indicating the hydrogen concentration in the space inside the toilet bowl 101 measured by the internal sensor 1 arranged inside the toilet bowl 101. The data acquisition unit 211 acquires the first time series data from the memory 22. The data acquisition unit 211 reads the first time series data stored in the memory 22.

The excreta determination unit 212 determines whether or not the one who excreted has farted based on the first time series data of the hydrogen concentration. When the value of the hydrogen concentration in the first time series data exceeds the threshold value, the excreta determination unit 212 determines that the one who excreted has farted.

The determination result output unit 213 outputs a determination result as to whether or not the one who excreted has farted. The determination result output unit 213 transmits determination result information indicating whether or not the one who excreted has farted to the server 3 via the communication unit 23.

Note that in a case where it is determined that the one who excreted has farted, the determination result output unit 213 may transmit the determination result information indicating that the one who excreted has farted and date and time information indicating the date and time when the one who excreted farted to the server 3 via the communication unit 23. When it is determined that the one who excreted has not farted, the determination result output unit 213 needs not transmit determination result information indicating that the one who excreted has not farted to the server 3.

The communication unit 23 transmits a determination result as to whether or not the one who excreted has farted to the server 3. The excreta determination device 2 is communicably connected to the server 3 via a network 4. The network 4 is the Internet, for example.

The server 3 receives determination result information indicating whether or not the one who excreted has farted that is transmitted by the excreta determination device 2. The server 3 may receive the determination result information indicating that the one who excreted has farted and the date and time information indicating the date and time when the one who excreted farted. The server 3 includes a database that stores identification information for identifying the room or the house in which the excreta determination device 2 is arranged, determination result information indicating that the one who excreted has farted, and date and time information indicating the date and time when the one who excreted farted in association with one another. The identification information may be identification information for identifying a resident (one who excreted) of the room or the house in which the excreta determination device 2 is arranged.

For example, the caregiver uses a database of the server 3 when creating monitoring data of the care receiver. That is, a terminal device used by the caregiver acquires. from the server 3, the determination result information and the date and time information corresponding to the identification information of the care receiver, and creates monitoring data of the care receiver. For example, the terminal device may create the number of times of farting in one day as the monitoring data, may create the number of times of farting in one week as the monitoring data, or may create the number of times of farting in one month as the monitoring data. Furthermore, for example, the terminal device may create the time of farting in one day as the monitoring data, may create the date and time of farting in one week as the monitoring data, or may create the date and time of farting in one month as the monitoring data.

Next, the excreta determination processing in the excreta determination device 2 according to the first embodiment of the present disclosure will be described.

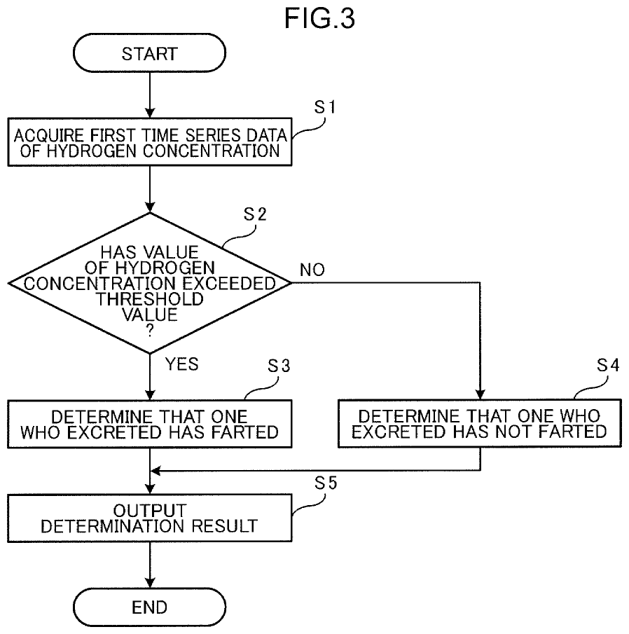

FIG. 3 is a flowchart for describing the excreta determination processing in the excreta determination device 2 according to the first embodiment of the present disclosure.

First, in step S1, the data acquisition unit 211 acquires, from the memory 22, the first time series data indicating the hydrogen concentration in the space inside the toilet bowl 101 measured by the internal sensor 1. For example, the excreta determination processing illustrated in FIG. 3 is performed once a day. The data acquisition unit 211 acquires the first time series data for one day at 0:00 AM, for example. Note that the time at which the first time series data is acquired is not limited to 0:00 AM. In addition, the excreta determination processing illustrated in FIG. 3 is not limited to be performed once a day, and may be performed a plurality of times a day, once a week, or every predetermined period.

In addition, the data acquisition unit 211 may acquire the first time series data in the period from the time point at which the one who excreted sits on the toilet seat 102 to the time point at which the one who excreted leaves the toilet seat 102. In a case where excreta is excreted a plurality of times in one day, the data acquisition unit 211 may acquire a plurality of first time series data for one day. Then, the excreta determination processing may be performed for each of the plurality of first time series data.

Next, in step S2, the excreta determination unit 212 determines whether or not the value of the hydrogen concentration in the first time series data has exceeded the threshold value. Here, when it is determined that the value of the hydrogen concentration has exceeded a threshold value (YES in step S2), the excreta determination unit 212 determines in step S3 that the one who excreted has farted. On the other hand, when it is determined that the value of the hydrogen concentration has not exceeded the threshold value (NO in step S2), the excreta determination unit 212 determines in step S4 that the one who excreted has not farted.

In the conventional technique, whether or not one who excreted has farted is discriminated based on temperature data, odor data of a hydrogen sulfide odor sensor, and odor data of an ammonia odor sensor. However, the concentrations of the hydrogen sulfide component and the ammonia component contained in the gas excreted from the anus of the one who excreted change depending on the physical condition such as constipation of the one who excreted. In addition, the concentrations of the hydrogen sulfide component and the ammonia component contained in the excreted gas greatly change depending on the food eaten by the one who excreted, and also greatly change depending on the medicine taken.

Regarding to this, the present inventors have found that the hydrogen concentration contained in the gas excreted from the anus of the human body is hardly affected by the food eaten by the human body and the medicine taken by the human body. Then, the present inventors have found that it is possible to determine whether or not the one who excreted has farted by measuring the time series change in the hydrogen concentration in the space inside the toilet bowl.

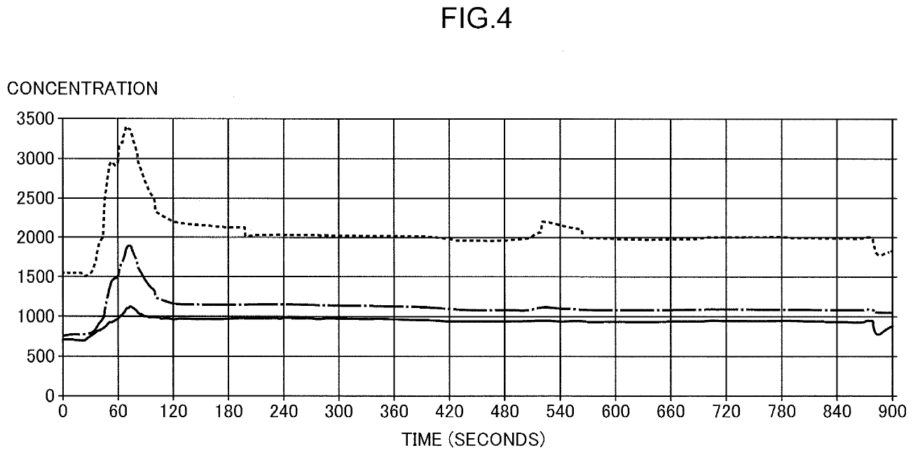

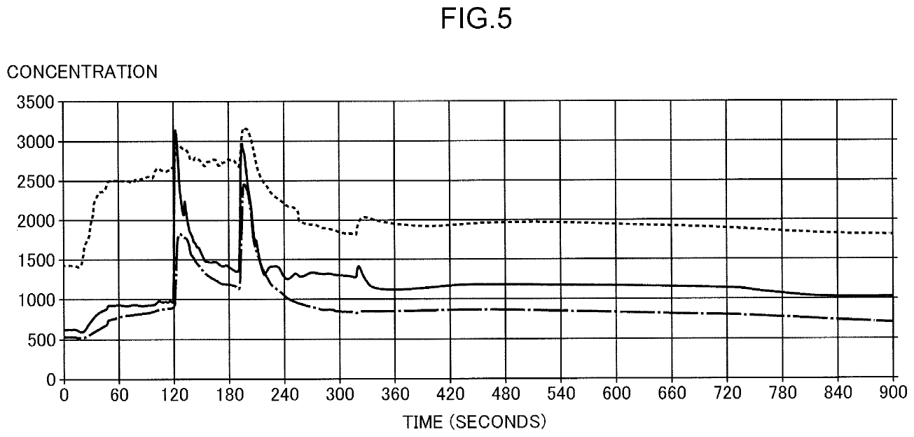

FIG. 4 is a view illustrating an example of first time series data of hydrogen concentration in a case where urination and defecation are present and fart is not present in the present first embodiment, and FIG. 5 is a view illustrating an example of first time series data of hydrogen concentration in a case where urination and fart are present and defecation is not present in the present first embodiment.

In FIGS. 4 and 5, the vertical axes represent the concentration (output value of the sensor) of each component, and the horizontal axes represent time (seconds). The internal sensor 1 may measure not only the hydrogen concentration but also the ammonia concentration and the hydrogen sulfide concentration. In FIGS. 4 and 5, solid lines represent the first time series data of the hydrogen concentration measured by the internal sensor 1, broken lines represent the time series data of the ammonia concentration measured by the internal sensor 1, and one-dot chain lines represent the time series data of the hydrogen sulfide concentration measured by the internal sensor 1.

In FIG. 4, the one who excreted sits on the toilet seat at the time point when about 15 seconds have elapsed, and urination and defecation are started at the time point when about 30 seconds have elapsed. With the start of urination and defecation, the ammonia concentration and the hydrogen sulfide concentration increase. On the other hand, the hydrogen concentration is hardly affected by urination and defecation, and exhibits a substantially constant value.

In FIG. 5, the one who excreted sits on the toilet seat at the time point when about 10 seconds have elapsed, and urination is started at the time point when about 20 seconds have elapsed. With the start of urination, the ammonia concentration increases. In addition, the first farting is performed at the time point when about 120 seconds have elapsed, and the second farting is performed at the time point when about 190 seconds have elapsed. The hydrogen concentration and the hydrogen sulfide concentration increase by the performance of farting.

When comparing the hydrogen concentrations in FIGS. 4 and 5, the hydrogen concentration greatly changes when farting is performed, and hardly changes when only urination and defecation are performed. From this, the excreta determination unit 212 can determine whether or not the one who excreted has farted by determining whether or not the value of the hydrogen concentration has exceeded the threshold value.

Returning to FIG. 3, next, in step S5, the determination result output unit 213 outputs a determination result as to whether or not the one who excreted has farted. For example, in a case where the excreta determination unit 212 determines that the one who excreted has farted, the determination result output unit 213 transmits, to the server 3 via the communication unit 23, the determination result information indicating that the one who excreted has farted and the date and time information indicating the date and time when the one who excreted farted.

Note that when the excreta determination unit 212 determines that the one who excreted has farted, the determination result output unit 213 may store, in the memory 22, the determination result information indicating that the one who excreted has farted and the date and time information indicating the date and time when the one who excreted farted. The excreta determination device 2 may include a universal serial bus (USB) port. The determination result output unit 213 may store the determination result information indicating that the one who excreted farted and the date and time information indicating the date and time when the one who excreted farted in a USB memory connected to the USB port.

Thus, the hydrogen concentration contained in the gas excreted from the anus of the human body is hardly affected by the food eaten by the human body and the medicine taken by the human body. Therefore, it is possible to accurately determine whether or not the one who excreted has farted by determining whether or not the one who excreted has farted based on the first time series data indicating the hydrogen concentration in the space inside the toilet bowl 101.

In the present first embodiment, the excreta determination unit 212 determines whether the value of the hydrogen concentration in the first time series data exceeds the threshold value, but the present disclosure is not particularly limited to this. The excreta determination unit 212 may determine that the one who excreted has farted when the value of the hydrogen concentration in the first time series data exceeds a threshold value, a rising slope of the first time series data until reaching a peak is larger than a threshold value, and a falling slope of the first time series data after reaching the peak is smaller than a threshold value.

As illustrated in FIG. 5, when the one who excreted farts, the hydrogen concentration rises sharply, reaches a peak, and then falls sharply. Therefore, the excreta determination unit 212 may determine whether or not the value of the hydrogen concentration in the first time series data has exceeded the threshold value. When determining that the value of the hydrogen concentration in the first time series data has exceeded the threshold value, the excreta determination unit 212 may determine whether or not the rising slope of the first time series data until reaching the peak is larger than a threshold value. Then, when determining that the rising slope of the first time series data until reaching the peak is larger than the threshold value, the excreta determination unit 212 may determine whether or not the falling slope of the first time series data after reaching the peak is smaller than a threshold value. Then, when determining that the falling slope of the first time series data after reaching the peak is smaller than the threshold value, the excreta determination unit 212 may determine that the one who excreted has farted. This makes it possible to more accurately determine whether or not the one who excreted has farted.

### Second Embodiment

The excreta determination device in the first embodiment acquires the first time series data indicating the hydrogen concentration in the space inside the toilet bowl measured by the internal sensor arranged inside the toilet bowl. On the other hand, the excreta determination device in the second embodiment acquires the first time series data indicating the hydrogen concentration in the space inside the toilet bowl measured by the internal sensor arranged inside the toilet bowl and the second time series data indicating the hydrogen concentration in the space outside the toilet bowl measured by the external sensor arranged outside the toilet bowl.

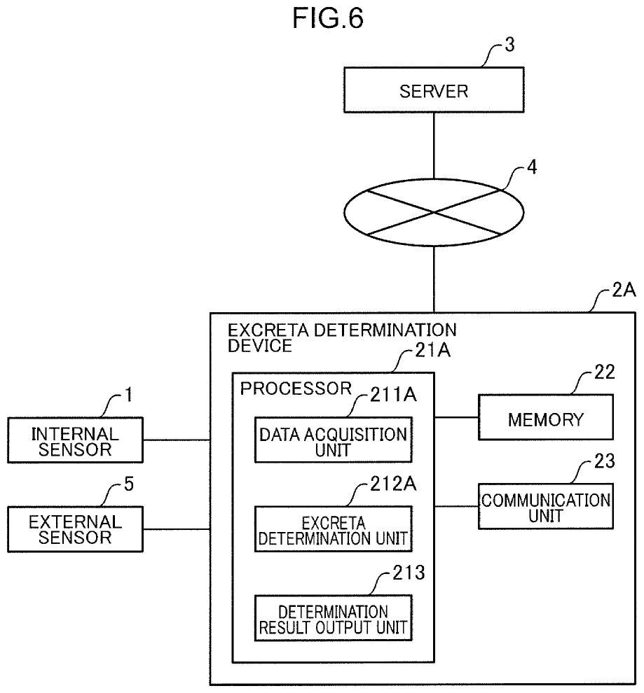

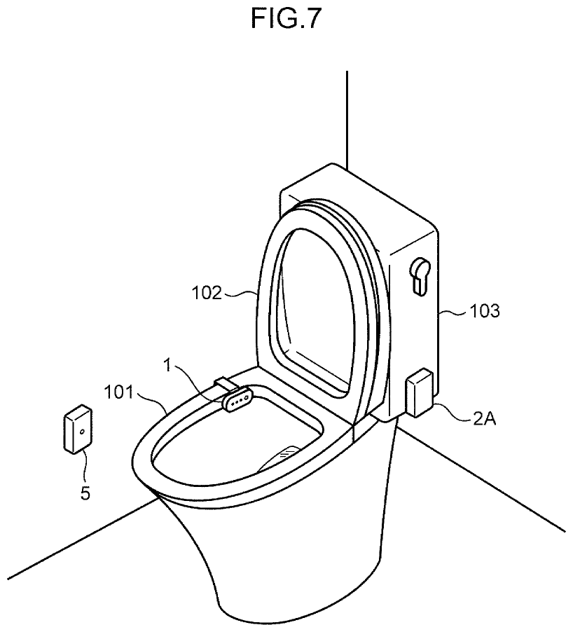

FIG. 6 is a view illustrating the configuration of an excretion management system in the second embodiment of the present disclosure. FIG. 7 is a view for describing the arrangement positions of the internal sensor 1, an external sensor 5, and an excreta determination device 2A in the second embodiment of the present disclosure.

The excretion management system illustrated in FIG. 6 includes the internal sensor 1, an excreta determination device 2A, the server 3, and an external sensor 5. In the present second embodiment, the same components as those in the first embodiment are given the same reference numerals, and description will be omitted.

The external sensor 5 is arranged outside the toilet bowl 101 and has sensitivity to hydrogen. As illustrated in FIG. 7, the external sensor 5 is attached to a wall surface in the toilet. The external sensor 5 measures the hydrogen concentration in the space outside the toilet bowl 101. The external sensor 5 is connected to the excreta determination device 2A communicably with each other in a wired or wireless manner. The external sensor 5 transmits the measured second time series data of the hydrogen concentration to the excreta determination device 2A.

Note that the arrangement position of the external sensor 5 is not limited to the wall surface inside the toilet, and may be anywhere as long as it is outside the toilet bowl 101 and inside the toilet. The external sensor 5 may constantly transmit the second time series data of the measured hydrogen concentration to the excreta determination device 2A. In addition, the external sensor 5 may transmit, to the excreta determination device 2A, the second time series data of the hydrogen concentration measured in a period from a time point at which the one who excreted sits on the toilet seat 102 to a time point at which the one who excreted leaves the toilet seat 102.

The excreta determination device 2A includes a processor 21A, the memory 22, and the communication unit 23.

The processor 21A is, for example, a CPU. The processor 21A implements a data acquisition unit 211A, an excreta determination unit 212A, and the determination result output unit 213.

The data acquisition unit 211A acquires the first time series data indicating the hydrogen concentration in the space inside the toilet bowl 101 measured by the internal sensor 1 arranged inside the toilet bowl 101. In addition, the data acquisition unit 211A acquires the second time series data indicating the hydrogen concentration in the space outside the toilet bowl 101 measured by the external sensor 5 arranged outside the toilet bowl 101. The data acquisition unit 211A acquires the first time series data and the second time series data from the memory 22. The data acquisition unit 211A reads the first time series data and the second time series data stored in the memory 22. The data acquisition unit 211A acquires the first time series data and the second time series data synchronized with the first time series data.

The excreta determination unit 212A determines whether or not the one who excreted has farted based on the first time series data and the second time series data. The excreta determination unit 212A determines that the one who excreted has farted when the value of the hydrogen concentration in the first time series data exceeds a threshold value and the value of the hydrogen concentration in the second time series data exceeds a threshold value.

Note that the threshold value to be compared with the value of the hydrogen concentration in the first time series data and the threshold value to be compared with the value of the hydrogen concentration in the second time series data may be the same. In addition, the threshold value to be compared with the value of the hydrogen concentration in the first time series data and the threshold value to be compared with the value of the hydrogen concentration in the second time series data may be different.

Subsequently, excreta determination processing in the excreta determination device 2A according to the second embodiment of the present disclosure will be described.

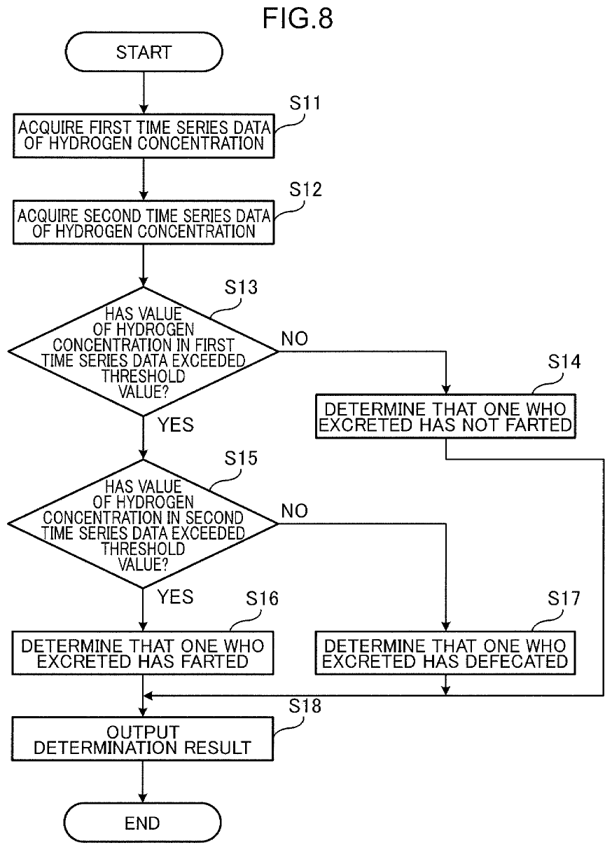

FIG. 8 is a flowchart for describing the excreta determination processing in the excreta determination device 2A according to the second embodiment of the present disclosure.

First, in step S11, the data acquisition unit 211A acquires, from the memory 22, the first time series data indicating the hydrogen concentration in the space inside the toilet bowl 101 measured by the internal sensor 1.

Next, in step S12, the data acquisition unit 211A acquires, from the memory 22, the second time series data indicating the hydrogen concentration in the space outside the toilet bowl 101 measured by the external sensor 5.

For example, the excreta determination processing illustrated in FIG. 8 is performed once a day. The data acquisition unit 211A acquires the first time series data and the second time series data for one day at 0:00 AM, for example. Note that the time at which the first time series data and the second time series data are acquired is not limited to 0:00 AM. In addition, the excreta determination processing illustrated in FIG. 8 is not limited to be performed once a day, and may be performed once a week or every predetermined period.

In addition, the data acquisition unit 211A may acquire the first time series data and the second time series data in the period from the time point at which the one who excreted sits on the toilet seat 102 to the time point at which the one who excreted leaves the toilet seat 102. In a case where excreta is excreted a plurality of times in one day, the data acquisition unit 211A may acquire a plurality of first time series data and a plurality of second time series data for one day. Then, the excreta determination processing may be performed for each of the plurality of first time series data and the plurality of second time series data.

Next, in step S13, the excreta determination unit 212A determines whether or not the value of the hydrogen concentration in the first time series data has exceeded the threshold value. Here, when determining that the value of the hydrogen concentration in the first time series data has not exceeded the threshold value (NO in step S13), the excreta determination unit 212A determines in step S14 that the one who excreted has not farted. On the other hand, when determining that the value of the hydrogen concentration in the first time series data has exceeded the threshold value (YES in step S13), the excreta determination unit 212A determines in step S15 whether or not the value of the hydrogen concentration in the second time series data has exceeded the threshold value.

Here, when determining that the value of the hydrogen concentration in the second time series data has exceeded the threshold value (YES in step S15), the excreta determination unit 212A determines in step S16 that the one who excreted has farted. On the other hand, when determining that the value of the hydrogen concentration in the second time series data has not exceeded the threshold value (NO in step S15), the excreta determination unit 212A determines in step S17 that the one who excreted has defecated.

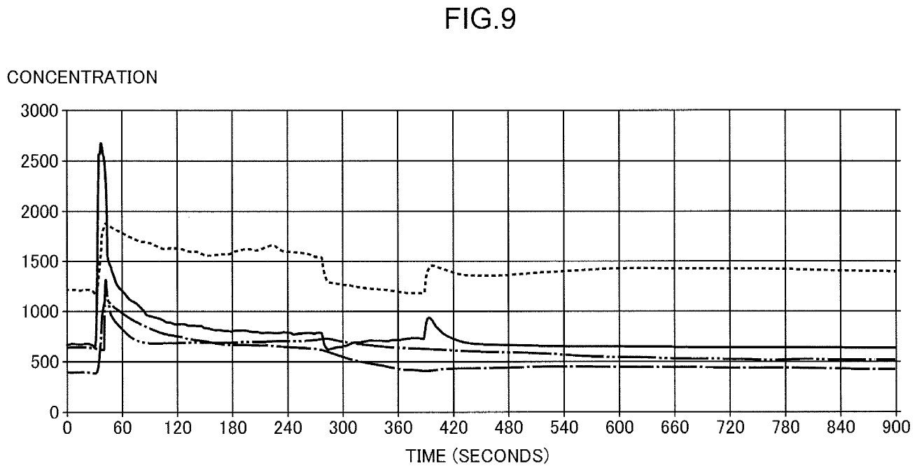

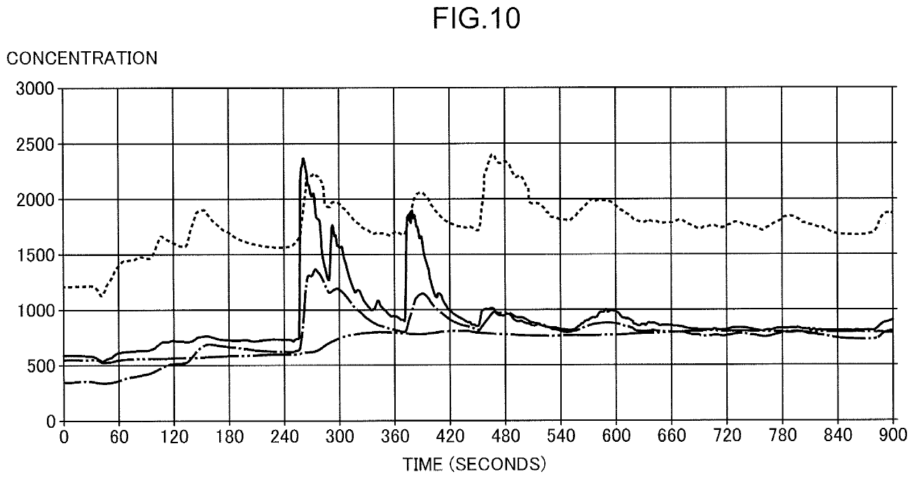

FIG. 9 is a view illustrating an example of first time series data and second time series data of hydrogen concentration in a case where urination and fart are present and defecation is not present in the present second embodiment, and FIG. 10 is a view illustrating an example of first time series data and second time series data of hydrogen concentration in a case where urination and defecation are present and fart is not present in the present second embodiment.

In FIGS. 9 and 10, the vertical axes represent the concentration (output value of the sensor) of each component, and the horizontal axes represent time (seconds). The internal sensor 1 may measure not only the hydrogen concentration but also the ammonia concentration and the hydrogen sulfide concentration. In FIGS. 9 and 10, solid lines represent the first time series data of the hydrogen concentration measured by the internal sensor 1, broken lines represent the time series data of the ammonia concentration measured by the internal sensor 1, one-dot chain lines represent the time series data of the hydrogen sulfide concentration measured by the internal sensor 1, and two-dot chain lines represent the second time series data of the hydrogen concentration measured by the external sensor 5.

In FIG. 9, the one who excreted sits on the toilet seat at the time point when about 20 seconds have elapsed, and urination is started at the time point when about 30 seconds have elapsed. With the start of urination, the ammonia concentration increases. In addition, farting is performed at the time point when about 30 seconds have elapsed. Due to the farting, the hydrogen concentration in the space inside the toilet bowl 101 increases. In addition, due to the farting, the hydrogen concentration in the space outside the toilet bowl 101 also increases. This is because the gas excreted from the anus of the one who excreted has diffused to the outside of the toilet bowl 101, and an increase in the hydrogen concentration due to the influence of the farting was observed also outside the toilet bowl 101 similarly to inside the toilet bowl 101.

Therefore, the excreta determination unit 212A determines that the one who excreted has farted when determining that the value of the hydrogen concentration in the first time series data has exceeded the threshold value and determines that the value of the hydrogen concentration in the second time series data has exceeded the threshold value. This makes it possible to more reliably determine that the one who excreted has farted.

In FIG. 10, on the other hand, the one who excreted sits on the toilet seat at the time point when about 30 seconds have elapsed, urination is started at the time point when about 40 seconds have elapsed, and defecation is started at the time point when about 260 seconds have elapsed. With the start of urination, the ammonia concentration increases, and with the start of defecation, the hydrogen sulfide concentration increases. At this time, the hydrogen concentration in the space inside the toilet bowl 101 increases with the start of defecation, but the hydrogen concentration in the space outside the toilet bowl 101 is hardly affected by the defecation and exhibits a substantially constant value. This enables the excreta determination unit 212A to determine that the one who excreted has defecated when determining that the value of the hydrogen concentration in the first time series data has exceeded the threshold value and determining that the value of the hydrogen concentration in the second time series data has not exceeded the threshold value.

Returning to FIG. 8, next, in step S18, the determination result output unit 213 outputs a determination result as to whether the one who excreted has farted or a determination result as to whether the one who excreted has defecated. For example, in a case where the excreta determination unit 212A determines that the one who excreted has farted, the determination result output unit 213 transmits, to the server 3 via the communication unit 23, the determination result information indicating that the one who excreted has farted and the date and time information indicating the date and time when the one who excreted farted. When the excreta determination unit 212A determines that the one who excreted has defecated, the determination result output unit 213 transmits, to the server 3 via the communication unit 23, determination result information indicating that the one who excreted has defecated and date and time information indicating the date and time when the one who excreted defecated.

Note that the excreta determination unit 212A may determine that the one who excreted has farted when the value of the hydrogen concentration in the first time series data exceeds a threshold value, a rising slope of the first time series data until reaching a peak is larger than a threshold value, a falling slope of the first time series data after reaching the peak is smaller than a threshold value, the value of the hydrogen concentration in the second time series data exceeds a threshold value, a rising slope of the second time series data until reaching a peak is larger than a threshold value, and a falling slope of the second time series data after reaching the peak is smaller than a threshold value.

### Third Embodiment

When the value of the hydrogen concentration in the first time series data exceeds the threshold value, the excreta determination device in the first embodiment determines that the one who excreted has farted. On the other hand, the excreta determination device in the third embodiment acquires the first time at which the first time series data reaches a peak and the second time at which the first time series data converges after the first time series data reached the peak, and determines which of feces or fart the one who excreted has excreted based on the first time and the second time.

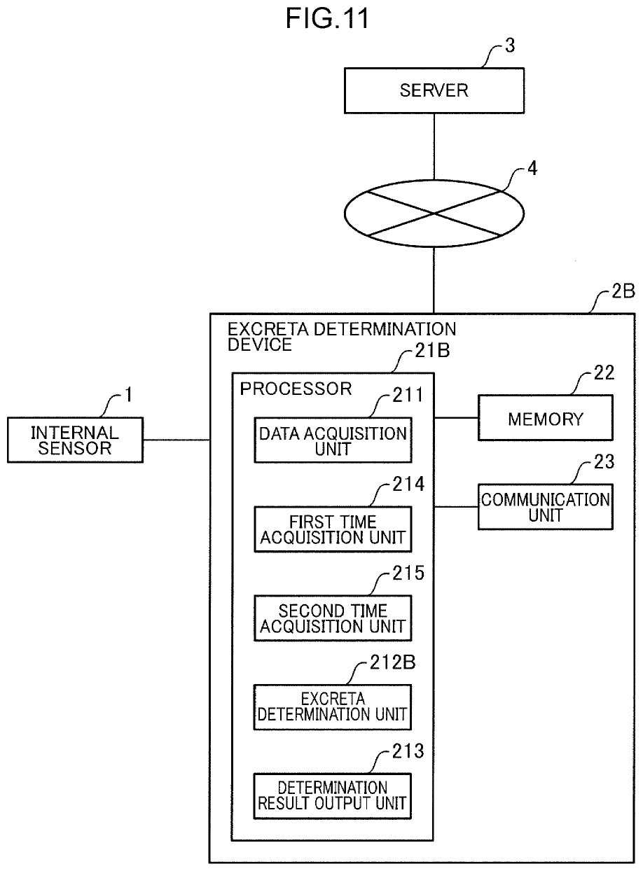

FIG. 11 is a view illustrating the configuration of an excretion management system in the third embodiment of the present disclosure. The arrangement positions of the internal sensor 1 and the excreta determination device 2B in the third embodiment of the present disclosure are the same as the arrangement positions of the internal sensor 1 and the excreta determination device 2 in the first embodiment.

The excretion management system illustrated in FIG. 11 includes the internal sensor 1, an excreta determination device 2B, and the server 3. In the present third embodiment, the same components as those in the first embodiment are given the same reference numerals, and description will be omitted.

The excreta determination device 2B includes a processor 21B, the memory 22, and the communication unit 23.

The processor 21B is, for example, a CPU. The processor 21B implements a data acquisition unit 211, an excreta determination unit 212B, the determination result output unit 213, a first time acquisition unit 214, and a second time acquisition unit 215.

The first time acquisition unit 214 acquires the first time at which the first time series data reached the peak.

The second time acquisition unit 215 acquires the second time at which the first time series data converged after the first time series data reached the peak.

The excreta determination unit 212B determines which of feces or fart the one who excreted has excreted based on the first time acquired by the first time acquisition unit 214 and the second time acquired by the second time acquisition unit 215. The excreta determination unit 212B determines that the one who excreted has farted when the difference between the second time and the first time is equal to or less than a predetermined time, and determines that the one who excreted has defecated when the difference is longer than the predetermined time.

Next, the excreta determination processing in the excreta determination device 2B according to the third embodiment of the present disclosure will be described.

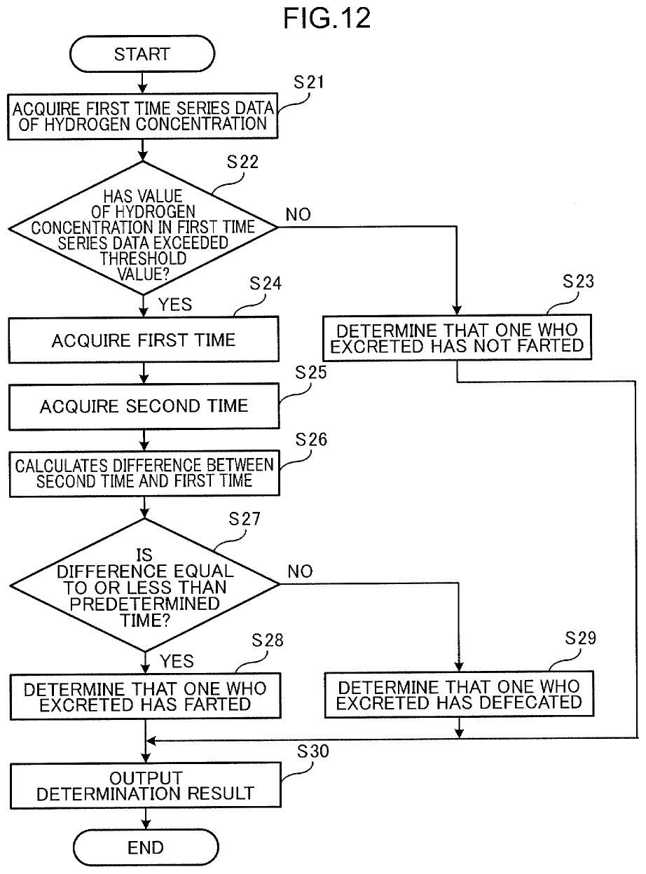

FIG. 12 is a flowchart for describing the excreta determination processing in the excreta determination device 2B according to the third embodiment of the present disclosure.

First, in step S21, the data acquisition unit 211 acquires, from the memory 22, the first time series data indicating the hydrogen concentration in the space inside the toilet bowl 101 measured by the internal sensor 1. For example, the excreta determination processing illustrated in FIG. 12 is performed once a day. The data acquisition unit 211 acquires the first time series data for one day at 0:00 AM, for example. Note that the time at which the first time series data is acquired is not limited to 0:00 AM. In addition, the excreta determination processing illustrated in FIG. 12 is not limited to be performed once a day, and may be performed once a week or every predetermined period.

In addition, the data acquisition unit 211 may acquire the first time series data in the period from the time point at which the one who excreted sits on the toilet seat 102 to the time point at which the one who excreted leaves the toilet seat 102. In a case where excreta is excreted a plurality of times in one day, the data acquisition unit 211 may acquire a plurality of first time series data for one day. Then, the excreta determination processing may be performed for each of the plurality of first time series data.

Next, in step S22, the excreta determination unit 212B determines whether or not the value of the hydrogen concentration in the first time series data has exceeded the threshold value. Here, when determining that the value of the hydrogen concentration in the first time series data has not exceeded the threshold value (NO in step S22), the excreta determination unit 212B determines in step S23 that the one who excreted has not farted.

On the other hand, when it is determined that the value of the hydrogen concentration in the first time series data has exceeded the threshold value (YES in step S22), the first time acquisition unit 214 acquires in step S24 the first time at which the first time series data acquired by the data acquisition unit 211 reaches the peak.

Next, in step S25, the second time acquisition unit 215 acquires the second time at which the first time series data acquired by the data acquisition unit 211 converged after the first time series data reached the peak.

Next, in step S26, the excreta determination unit 212B calculates a difference between the second time and the first time.

Next, in step S27, the excreta determination unit 212B determines whether or not the difference is equal to or less than a predetermined time.

Here, when determining that the difference is equal to or less than the predetermined time (YES in step S27), the excreta determination unit 212B determines in step S28 that the one who excreted has farted.

On the other hand, when determining that the difference is longer than the predetermined time (NO in step S27), the excreta determination unit 212B determines in step S29 that the one who excreted has defecated.

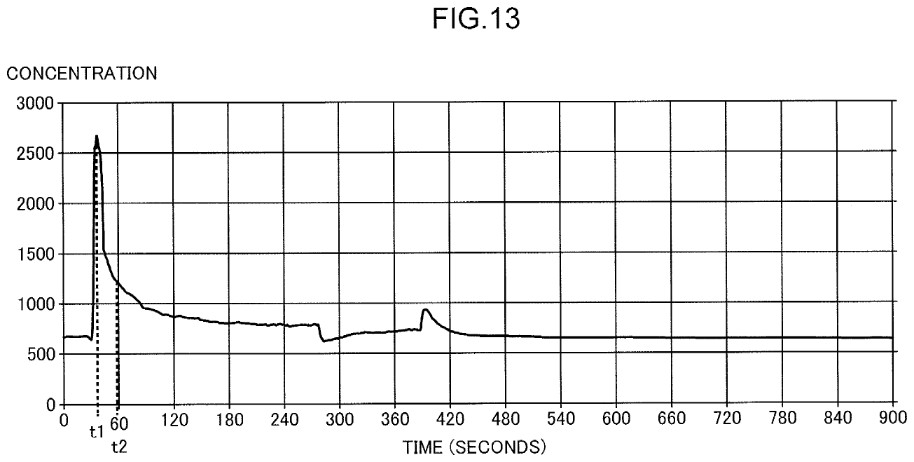

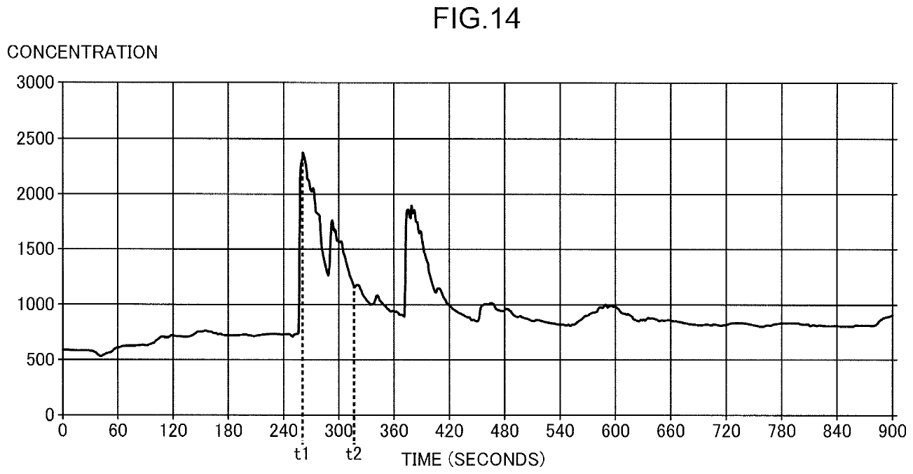

FIG. 13 is a view illustrating an example of first time series data of hydrogen concentration in a case where farting is present and defecation is not present in the present third embodiment, and FIG. 14 is a view illustrating an example of first time series data of hydrogen concentration in a case where defecation is present and farting is not present in the present third embodiment.

In FIGS. 13 and 14, the vertical axes represent the hydrogen concentration (output value of the sensor), and the horizontal axes represent time (seconds). In FIGS. 13 and 14, solid lines represent the first time series data of the hydrogen concentration measured by the internal sensor 1.

In FIG. 13, the one who excreted sits on the toilet seat at the time point when about 20 seconds have elapsed, and farting is performed at the time point when about 30 seconds have elapsed. When farting is performed, the hydrogen concentration in the space inside the toilet bowl 101 rises sharply, reaches a peak, and then falls sharply. This is because the gas excreted from the anus of the one who excreted is diffused. When the one who excreted farts, the period from first time t1 at which the value of the hydrogen concentration in the space inside the toilet bowl 101 reaches the peak to second time t2 at which the value of the hydrogen concentration converges is about 20 seconds. For example, the time point at which the decrease amount from the peak value becomes 80% of the increase amount from the rise to the peak is defined as the second time at which the value of the hydrogen concentration converges.

In FIG. 14, on the other hand, the one who excreted sits on the toilet seat at the time point when about 30 seconds have elapsed, and defecation is started at the time point when about 260 seconds have elapsed. When defecation is performed, the hydrogen concentration in the space inside the toilet bowl 101 rises sharply, reaches a peak, and then gradually decreases. This is because feces exist inside the toilet bowl 101. When the one who excreted defecates, the period from first time t1 at which the value of the hydrogen concentration in the space inside the toilet bowl 101 reaches the peak to second time t2 at which the value of the hydrogen concentration converges is about 60 seconds.

From the above, the elapsed time from when the hydrogen concentration reaches the peak to when it converges is different between fart and defecation. Therefore, by the elapsed time from when the value of the hydrogen concentration reaches the peak to when it converges, it is possible to determine which of feces and fart the one who excreted has excreted. When the difference between the second time and the first time is, for example, equal to or less than 30 seconds, the excreta determination unit 212B may determine that the one who excreted has farted. When the difference between the second time and the first time is longer than, for example, 30 seconds, the excreta determination unit 212B may determine that the one who excreted has defecated.

Returning to FIG. 12, next, in step S30, the determination result output unit 213 outputs a determination result as to whether the one who excreted has farted or a determination result as to whether the one who excreted has defecated. For example, in a case where the excreta determination unit 212B determines that the one who excreted has farted, the determination result output unit 213 transmits, to the server 3 via the communication unit 23, the determination result information indicating that the one who excreted has farted and the date and time information indicating the date and time when the one who excreted farted. When the excreta determination unit 212B determines that the one who excreted has defecated, the determination result output unit 213 transmits, to the server 3 via the communication unit 23, determination result information indicating that the one who excreted has defecated and date and time information indicating the date and time when the one who excreted defecated.

### Fourth Embodiment

The excreta determination device in the third embodiment determines which of feces or fart the one who excreted has excreted based on the first time at which the first time series data reaches a peak and the second time at which the first time series data converges after the first time series data reached the peak. On the other hand, the excreta determination device in the fourth embodiment acquires the first time series data indicating the hydrogen concentration in the space inside the toilet bowl measured by the internal sensor arranged inside the toilet bowl and the second time series data indicating the hydrogen concentration in the space outside the toilet bowl measured by the external sensor arranged outside the toilet bowl, and determines which of feces or fart the one who excreted has excreted based on the first time at which the first time series data reaches a peak, the second time at which the first time series data converges after the first time series data reached the peak, the third time at which the second time series data reaches a peak, and the fourth time at which the second time series data converges after the second time series data reached the peak.

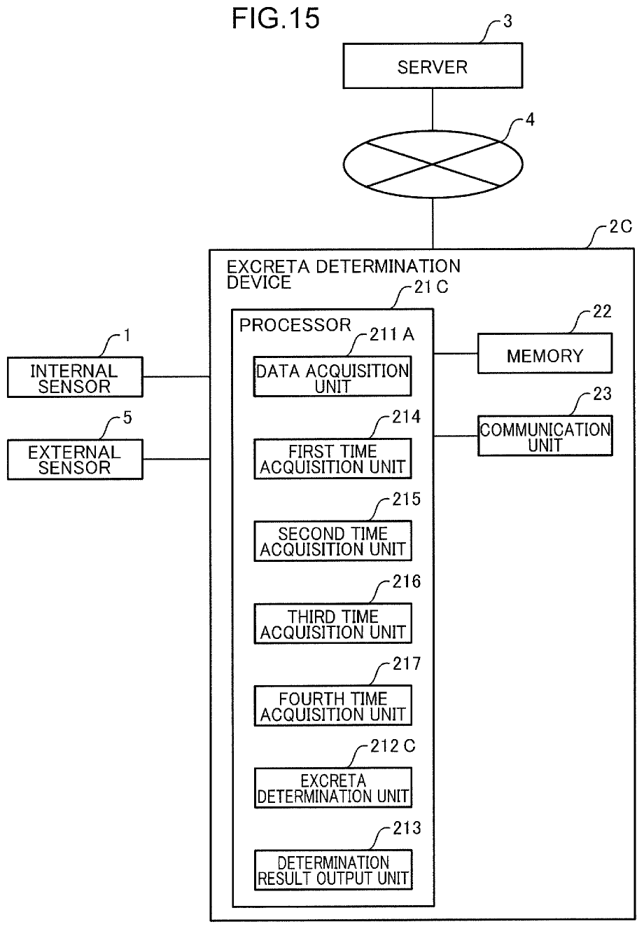

FIG. 15 is a view illustrating the configuration of an excretion management system in the fourth embodiment of the present disclosure. The arrangement positions of the internal sensor 1, the external sensor 5, and an excreta determination device 2C in the fourth embodiment of the present disclosure are the same as the arrangement positions of the internal sensor 1, the external sensor 5, and the excreta determination device 2A in the second embodiment.

The excretion management system illustrated in FIG. 15 includes the internal sensor 1, the external sensor 5, the excreta determination device 2C, and the server 3. In the present fourth embodiment, the same components as those in the first to third embodiments are given the same reference numerals, and description will be omitted.

The excreta determination device 2C includes a processor 21C, the memory 22, and the communication unit 23.

The processor 21C is, for example, a CPU. The processor 21C implements the data acquisition unit 211A, an excreta determination unit 212C, the determination result output unit 213, the first time acquisition unit 214, the second time acquisition unit 215, a third time acquisition unit 216, and a fourth time acquisition unit 217.

The third time acquisition unit 216 acquires the third time at which the second time series data reaches the peak.

The fourth time acquisition unit 217 acquires the fourth time at which the second time series data converges after the second time series data reaches the peak.

The excreta determination unit 212C determines which of feces or fart the one who excreted has excreted based on the first time acquired by the first time acquisition unit 214, the second time acquired by the second time acquisition unit 215, the third time acquired by the third time acquisition unit 216, and the fourth time acquired by the fourth time acquisition unit 217. The excreta determination unit 212C determines that the one who excreted has farted when the first difference between the second time and the first time is equal to or less than a predetermined time and the second difference between the fourth time and the third time is equal to or less than a predetermined time, and determines that the one who excreted has defecated when the first difference is longer than the predetermined time and the second difference is longer than the predetermined time.

Note that the predetermined time to be compared with the first difference and the predetermined time to be compared with the second difference may be the same. The predetermined time to be compared with the first difference and the predetermined time to be compared with the second difference may be different.

Next, the excreta determination processing in the excreta determination device 2C according to the fourth embodiment of the present disclosure will be described.

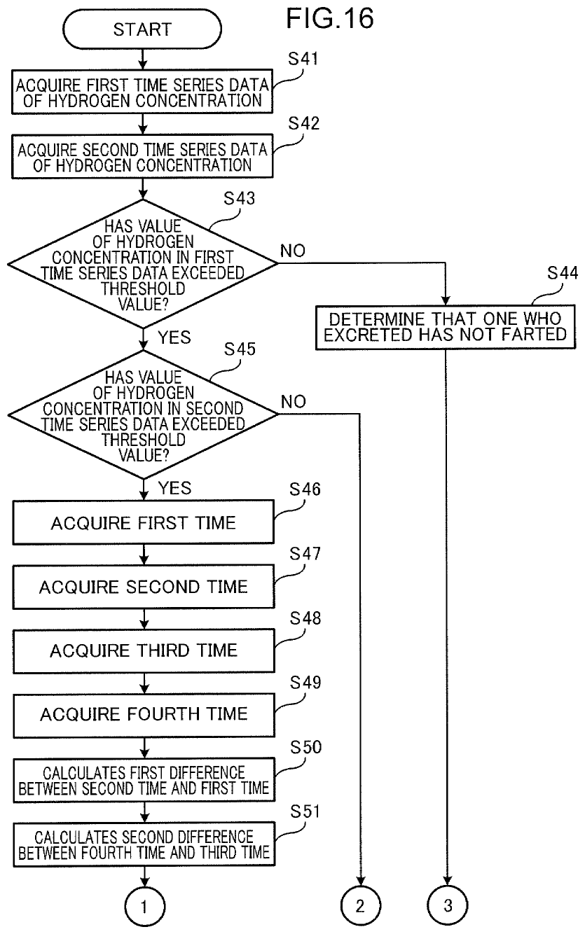

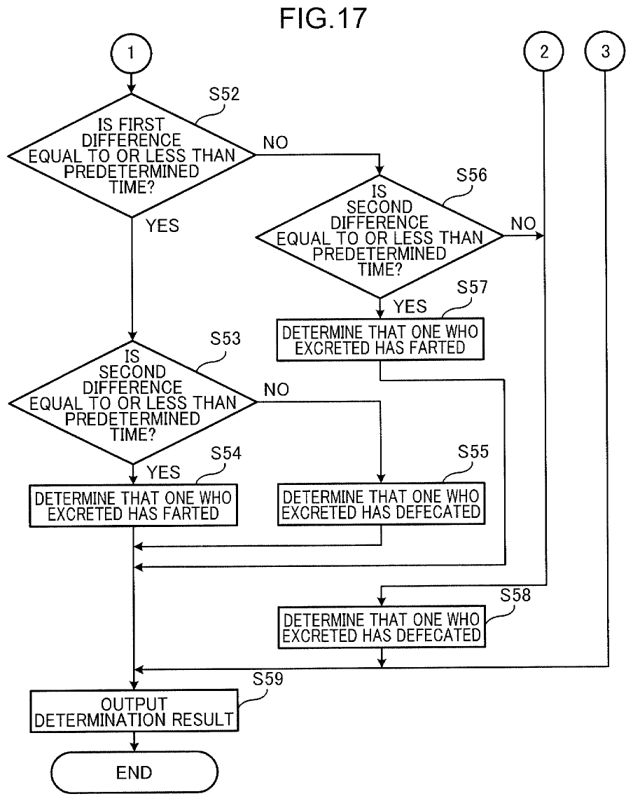

FIG. 16 is the first flowchart for describing the excreta determination processing in the excreta determination device 2C according to the fourth embodiment of the present disclosure, and FIG. 17 is a second flowchart for describing the excreta determination processing in the excreta determination device 2C according to the fourth embodiment of the present disclosure.

Note that the processing in steps S41 to S45 is the same as the processing in steps S11 to S15 in FIG. 8, and thus description is omitted.

When determining that the value of the hydrogen concentration in the second time series data has not exceeded the threshold value (NO in step S45), the excreta determination unit 212C determines in step S58 that the one who excreted has defecated.

On the other hand, when it is determined that the value of the hydrogen concentration in the second time series data has exceeded the threshold value (YES in step S45), the first time acquisition unit 214 acquires in step S46 the first time at which the first time series data acquired by the data acquisition unit 211A reached the peak.

Next, in step S47, the second time acquisition unit 215 acquires the second time at which the first time series data acquired by the data acquisition unit 211A converged after the first time series data reached the peak.

Next, in step S48, the third time acquisition unit 216 acquires the third time at which the second time series data acquired by the data acquisition unit 211A reaches the peak.

Next, in step S49, the fourth time acquisition unit 217 acquires the fourth time at which the second time series data acquired by the data acquisition unit 211A converges after the second time series data reaches the peak.

Next, in step S50, the excreta determination unit 212C calculates the first difference between the second time and the first time.

Next, in step S51, the excreta determination unit 212C calculates the second difference between the fourth time and the third time.

Next, in step S52, the excreta determination unit 212C determines whether or not the first difference is equal to or less than a predetermined time.

Here, when determining that the first difference is equal to or less than the predetermined time (YES in step S52), the excreta determination unit 212C determines in step S53 whether or not the second difference is equal to or less than a predetermined time. When determining that the second difference is equal to or less than the predetermined time (YES in step S53), the excreta determination unit 212C determines in step S54 that the one who excreted has farted. On the other hand, when determining that the second difference is longer than the predetermined time (NO in step S53), the excreta determination unit 212C determines in step S55 that the one who excreted has defecated.

When determining that the first difference is longer than the predetermined time (NO in step S52), the excreta determination unit 212C determines in step S56 whether or not the second difference is equal to or less than the predetermined time. When determining that the second difference is equal to or less than the predetermined time (YES in step S56), the excreta determination unit 212C determines in step S57 that the one who excreted has farted. On the other hand, when determining that the second difference is longer than the predetermined time (NO in step S56), the excreta determination unit 212C determines in step S58 that the one who excreted has defecated.

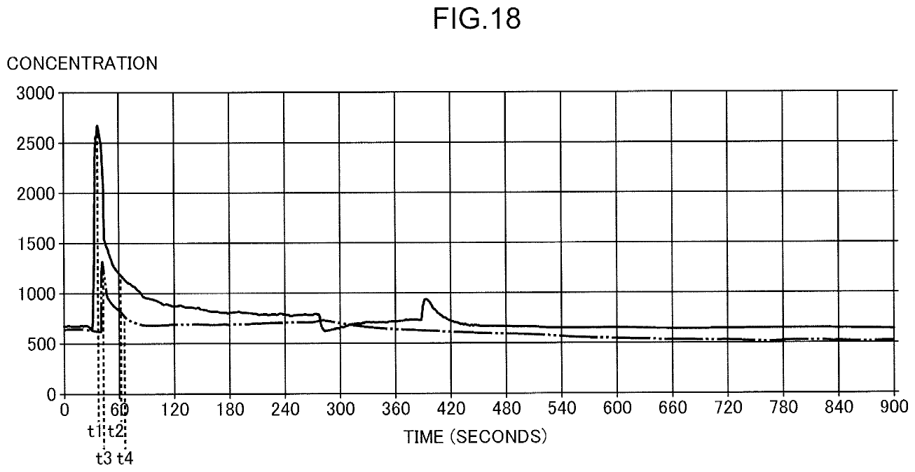

FIG. 18 is a view illustrating an example of first time series data of hydrogen concentration in a case where farting is present and defecation is not present in the present fourth embodiment.

In FIG. 18, the vertical axis represents the hydrogen concentration (output value of the sensor), and the horizontal axis represents time (seconds). In FIG. 18, a solid line represents the first time series data of the hydrogen concentration measured by the internal sensor 1, and a two-dot chain line represents the second time series data of the hydrogen concentration measured by the external sensor 5.

In FIG. 18, the one who excreted sits on the toilet seat at the time point when about 20 seconds have elapsed, and farting is performed at the time point when about 30 seconds have elapsed. When farting is performed, the hydrogen concentration in the space inside the toilet bowl 101 rises sharply, reaches a peak, and then falls sharply. This is because the gas excreted from the anus of the one who excreted is diffused. When the one who excreted farts, the period from first time t1 at which the value of the hydrogen concentration in the space inside the toilet bowl 101 reaches the peak to second time t2 at which the value of the hydrogen concentration converges is about 20 seconds. For example, the time point at which the decrease amount from the peak value becomes 80% of the increase amount from the rise to the peak is defined as the second time at which the value of the hydrogen concentration converges.

In addition, due to the farting, the hydrogen concentration in the space outside the toilet bowl 101 also increases. This is because the gas excreted from the anus of the one who excreted has diffused to the outside of the toilet bowl 101, and an increase in the hydrogen concentration due to the influence of the farting was observed also outside the toilet bowl 101 similarly to inside the toilet bowl 101. When farting is performed, the hydrogen concentration in the space outside the toilet bowl 101 also rises sharply, reaches a peak, and then falls sharply. When the one who excreted farts, the period from third time t3 at which the value of the hydrogen concentration in the space outside the toilet bowl 101 reaches the peak to fourth time t4 at which the value of the hydrogen concentration converges is about 20 seconds.

It is possible to more accurately determine which of feces or fart the one who excreted has excreted by the elapsed time from when the value of hydrogen concentration in the space inside the toilet bowl 101 reaches the peak to when it converges and the elapsed time from when the value of hydrogen concentration in the space outside the toilet bowl 101 reaches the peak to when it converges. That is, when the first difference between the second time and the first time is, for example, equal to or less than 30 seconds and the second difference between the fourth time and the third time is, for example, equal to or less than 30 seconds, the excreta determination unit 212C may determine that the one who excreted has farted.

When the first difference between the second time and the first time is longer than, for example, 30 seconds and the second difference between the fourth time and the third time is longer than, for example, 30 seconds, the excreta determination unit 212C may determine that the one who excreted has defecated.

As described above, by using not only the measurement data of the internal sensor 1 but also the measurement data of the external sensor 5, it is possible to more accurately determine which of fart and feces the one who excreted has excreted.

Furthermore, when the first difference between the second time and the first time is, for example, equal to or less than 30 seconds and the second difference between the fourth time and the third time is longer than, for example, 30 seconds, the excreta determination unit 212C may determine that the one who excreted has defecated. When the first difference between the second time and the first time is longer than, for example, 30 seconds and the second difference between the fourth time and the third time is, for example, equal to or less than 30 seconds, the excreta determination unit 212C may determine that the one who excreted has farted.

As a result, for example, even if erroneous data is measured by the internal sensor 1, correct determination can be made using the data of the external sensor 5.

Note that there is a concern that the measurement data of the external sensor 5 is affected by a fragrance provided in the toilet or a cleaning agent used for cleaning the toilet. Therefore, when the comparison result of the first difference and the comparison result of the second difference are different, the excreta determination unit 212C may give priority to the comparison result of the first difference. For example, the excreta determination unit 212C may determine that the one who excreted has farted when determining that the first difference is equal to or less than a predetermined time and the second difference is longer than a predetermined time (YES in step S52 and NO in step S53). For example, when determining that the first difference is longer than the predetermined time and the second difference is equal to or less than the predetermined time (NO in step S52 and YES in step S56), the excreta determination unit 212C may determine that the one who excreted has defecated. Due to this, even in a case where the accuracy of the measurement data of the external sensor 5 is low, it is possible to accurately determine which of fart and feces the one who excreted has excreted.

Returning to FIG. 17, next, in step S59, the determination result output unit 213 outputs a determination result as to whether the one who excreted has farted or a determination result as to whether the one who excreted has defecated. For example, in a case where the excreta determination unit 212C determines that the one who excreted has farted, the determination result output unit 213 transmits, to the server 3 via the communication unit 23, the determination result information indicating that the one who excreted has farted and the date and time information indicating the date and time when the one who excreted farted. When the excreta determination unit 212C determines that the one who excreted has defecated, the determination result output unit 213 transmits, to the server 3 via the communication unit 23, determination result information indicating that the one who excreted has defecated and date and time information indicating the date and time when the one who excreted defecated.

In the present first to fourth embodiments, only one person uses one toilet, but the present disclosure is not particularly limited to this, and a plurality of persons may use one toilet. In this case, the excretion management system may further include a recognition device for recognizing a person who uses the toilet. The recognition device is, for example, a camera, and may image the face of the person who uses the toilet and recognize the person who uses the toilet from the face image obtained by the imaging.

In each of the above embodiments, each component may be implemented by being configured with dedicated hardware or by executing a software program suitable for each component. Each component may be implemented by a program execution unit such as a CPU or a processor reading and executing a software program recorded in a recording medium such as a hard disk or a semiconductor memory. In addition, the program may be carried out by another independent computer system by recording and transferring the program onto a recording medium or transferring the program via a network.

Some or all of the functions of the devices according to the embodiments of the present disclosure are implemented as large scale integration (LSI), which is typically an integrated circuit. These may be individually integrated into one chip, or may be integrated into one chip so as to include some or all of the functions. Furthermore, the circuit integration is not limited to LSI, and may be implemented by a dedicated circuit or a general-purpose processor. A field programmable gate array (FPGA) that can be programmed after manufacturing of LSI or a reconfigurable processor in which connections and settings of circuit cells inside LSI can be reconfigured may be used.

Some or all of the functions of the devices according to the embodiments of the present disclosure may be implemented by execution of a program by a processor such as a CPU.

In addition, the numbers used above are all illustrated to specifically describe the present disclosure, and the present disclosure is not limited to the illustrated numbers.

In addition, the order in which each step illustrated in the above flowcharts is executed is for specifically describing the present disclosure, and may be an order other than the above order as long as a similar effect is obtained. In addition, some of the above steps may be executed simultaneously (concurrently) with other steps.

### INDUSTRIAL APPLICABILITY

Since the technique according to the present disclosure, it is possible to accurately determine whether or not one who excreted has farted, the technique according to the present disclosure is useful as a technique for determining excreta.

1. An excreta determination method performed by a computer, the method comprising:
acquiring first time series data indicating hydrogen concentration in a space inside a toilet bowl measured by an internal sensor arranged inside the toilet bowl;
determining whether or not one who excreted has farted based on the first time series data of the hydrogen concentration; and
outputting a determination result.

2. The excreta determination method according to claim 1, wherein in the determination, when a value of the hydrogen concentration in the first time series data exceeds a threshold value, it is determined that the one who excreted has farted.

3. The excreta determination method according to claim 1, wherein in the determination, it is determined that the one who excreted has farted when a value of the hydrogen concentration in the first time series data exceeds a threshold value, a rising slope of the first time series data until reaching a peak is larger than a threshold value, and a falling slope of the first time series data after reaching the peak is smaller than a threshold value.

4. The excreta determination method according to claim 1, further comprising
acquiring second time series data indicating hydrogen concentration in a space outside the toilet bowl measured by an external sensor arranged outside the toilet bowl, and
wherein in the determination, it is determined whether or not the one who excreted has farted based on the first time series data and the second time series data.

5. The excreta determination method according to claim 4, wherein in the determination, it is determined that the one who excreted has farted when a value of the hydrogen concentration in the first time series data exceeds a threshold value and a value of the hydrogen concentration in the second time series data exceeds a threshold value.

6. The excreta determination method according to claim 1, further comprising:
acquiring first time at which the first time series data reaches a peak; and
acquiring second time at which the first time series data converges after the first time series data reached the peak, and
wherein in the determination, it is determined which of feces or fart the one who excreted has excreted based on the first time and the second time.

7. The excreta determination method according to claim 6, wherein in the determination, it is determined that the one who excreted has farted when a difference between the second time and the first time is equal to or less than a predetermined time, and it is determined that the one who excreted has defecated when the difference is longer than the predetermined time.

8. The excreta determination method according to claim 6, further comprising:
acquiring second time series data indicating hydrogen concentration in a space outside the toilet bowl measured by an external sensor arranged outside the toilet bowl;
acquiring third time at which the second time series data reaches a peak; and
acquiring fourth time at which the second time series data converges after the second time series data reached the peak, and
wherein in the determination, it is determined which of feces or fart the one who excreted has excreted based on the first time, the second time, the third time, and the fourth time.

9. The excreta determination method according to claim 8, wherein in the determination, it is determined that the one who excreted has farted when a first difference between the second time and the first time is equal to or less than a predetermined time and a second difference between the fourth time and the third time is equal to or less than a predetermined time, and it is determined that the one who excreted has defecated when the first difference is longer than the predetermined time and the second difference is longer than the predetermined time.

10. An excreta determination device comprising:
an acquisition unit that acquires first time series data indicating hydrogen concentration in a space inside a toilet bowl measured by an internal sensor arranged inside the toilet bowl;
a determination unit that determines whether or not one who excreted has farted based on the first time series data of the hydrogen concentration; and
an output unit that outputs a determination result.

11. A non-transitory computer readable recording medium storing an excreta determination program causing a computer to function to:
acquire first time series data indicating hydrogen concentration in a space inside a toilet bowl measured by an internal sensor arranged inside the toilet bowl;
determine whether or not one who excreted has farted based on the first time series data of the hydrogen concentration; and
output a determination result.
